(19) United States
(12) Patent Application Publication
(10) Pub. No.: US 2002/0031461 A1
(43) Pub. Date: Mar. 14, 2002

(54) The antibacterial toothbrush spray

(76) Inventor: Karri Lyn Knipp, Peoria, AZ (US)

Correspondence Address: Karri Knipp, 21832 N. 87th Ave., Peoria, AZ, 85382, US

(21) Appl. No.: 09/827,113

(22) Filed: Apr. 5, 2001

Related U.S. Application Data
(60) Provisional application No. 60/195,287, filed on Apr. 7, 2000.

(51) Int. Cl. A61L 2/00

(52) U.S. Cl. 422/300

(57) ABSTRACT

The Antibacterial Toothbrush Spray invention represents a newly designed toothbrush sanitizer. It will be composed of a plastic container designed to house several toothbrushes, covered by a dome-shaped lid with an incorporated pump. From an internal reservoir, spray disinfectant is pumped onto the surface of the toothbrushes through aligned spray holes. This product will provide users with a convenient and easy-to-use method of sanitizing their toothbrushes, thus killing germs and helping to prevent the spread of germs and illness.

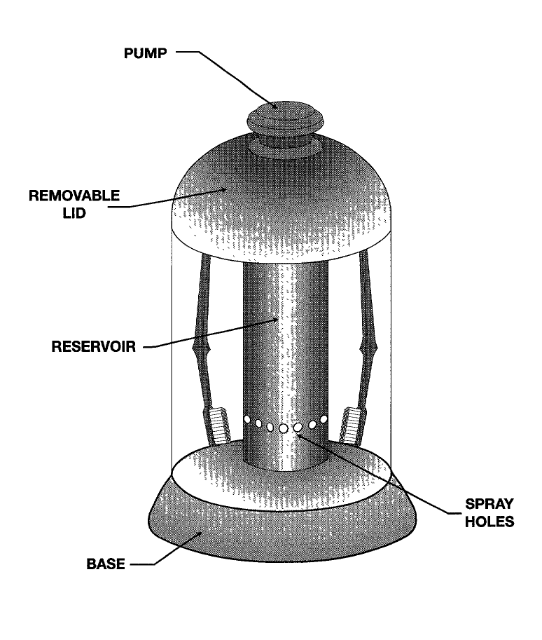

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

The toothbrush routinely comes into contact with bacteria, and often with viruses; yet it is rarely cleaned or sanitized after such contact. The Antibacterial Toothbrush Spray was created to address this problem. It was designed to provide a quick and simple technique for the routine application of antibacterial/antiviral solution to the contaminated surfaces of the toothbrush.

### BRIEF SUMMARY OF THE INVENTION

By disinfecting the toothbrush, the Antibacterial Toothbrush Spray has the potential to dramatically reduce the chances of re-introducing bacteria to the mouth that may otherwise linger on the toothbrush during an illness. During healthy periods, the Antibacterial Toothbrush Spray provides a convenient method for the general improvement of oral hygiene practices. In addition, there may be a reduced risk of cross-infection between a sick family member and those who store their toothbrushes within close proximity of the infected instrument.

### BRIEF DESCRIPTION OF THE SEVERAL VIEWS OF THE DRAWING

The Antibacterial Toothbrush Spray is comprised of a cylindrical plastic container, atop a slightly wider base structure, and covered by a removable, dome-shaped lid. Several toothbrushes, held in place by incorporated toothbrush holders, may be placed in the container simultaneously. A pump mechanism, activated by depressing the apical portion of the container's lid, draws antibacterial/antiviral solution from an internal reservoir located in the middle portion of the central column of the container. The solution is then released by spray from small openings in the lower portion of the central column. These openings are aligned with the brush ends of the toothbrushes.

### DETAILED DESCRIPTION OF THE INVENTION

This invention addresses the need for a convenient method of sanitation for the otherwise germ-laden toothbrush. The product will be constructed of plastic material, in conformation with the enclosed drawing. There are currently no similar devices with which to compare this invention; thus it is considered an improvement in the general area of oral hygiene, but not an improvement upon a previous invention.

The mode chosen for implementation of this invention is the marketing of information and drawings to manufacturers who may have an interest in the product. The inventor does not intend to fabricate the product or market the finalized product independently.

The anticipated target markets of the Antibacterial Toothbrush Spray include individual or family home use, group home use (e.g., elderly-housing centers, dormitories, assisted-living centers), medical or dental service providers, and specialty hospitals. ++ Specific, daily use of this product involves placement of the toothbrush, via the incorporated toothbrush holders, into the cylinder of the Antibacterial Toothbrush Spray, which sits atop its base. The removable lid is then placed on top of the cylinder. The pump at the apical portion of the removable lid is then depressed, which forces antibacterial/antiviral solution to spray from the reservoir through the spray holes over the lower surfaces of the toothbrush. The internal reservoir must be filled manually prior to first use and periodically for maintenance.

1. An oral hygiene device for the sanitation of toothbrushes comprising:
(a) a container composed of rigid material of sufficient size to hold a plurality of toothbrushes
(b) a base, upon which said container is permanently attached, to stabilize the unit
(c) an inner compartment, of which the upper portion is a reservoir and the lower portion contains spray holes
(d) a removable lid
(e) a pump mechanism, integrated within said removable lid, which draws from said reservoir
